(19) United States
(12) Patent Application Publication
(10) Pub. No.: US 2003/0152565 A1
(43) Pub. Date: Aug. 14, 2003

(54) Pharmaceutical compositions containing proteins

(75) Inventor: Alberto Bartorelli, Crans Sur Sierre (CH)

Correspondence Address: YOUNG & THOMPSON, 745 SOUTH 23RD STREET 2ND FLOOR, ARLINGTON, VA, 22202

(73) Assignee: PHARMAPRODUCTS UK LIMITED, LIVERPOOL MERSEYSIDE (GB)

(21) Appl. No.: 10/370,582

(22) Filed: Feb. 24, 2003

Related U.S. Application Data
(62) Division of application No. 09/857,375, filed on Sep. 25, 2001.
Application No. 09/857,375 is a 371 of international application No. PCT/EP99/09398, filed on Dec. 2, 1999.

(30) Foreign Application Priority Data
Dec. 4, 1998 (IT) MI98A002634

(51) Int. Cl. A61K 38/51

(52) U.S. Cl. 424/94.5

(57) ABSTRACT

A pharmaceutical composition with anti-tumor effect, consisting essentially of purified protein-disulfide isomerase in admixture with a pharmaceutically acceptable excipient. The isomerase is selected from the group consisting of ERp72, ERp60, P5, and calsequestrin. A method of combating tumors in humans, comprises administering to a human in need thereof a pharmaceutically effective amount of purified protein-disulfide isomerase in admixture with a pharmaceutically acceptable excipient, the effective amount being 1-10 mg.

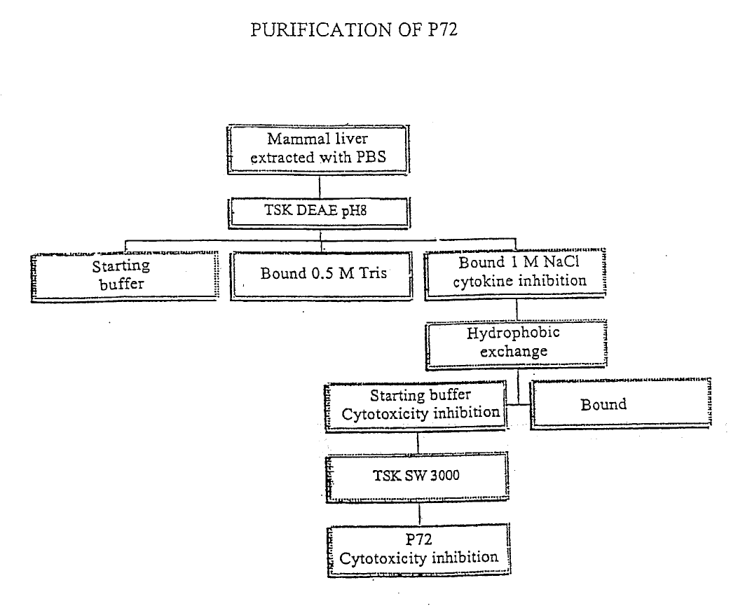

The present invention relates to the use of proteins known as protein-disulfide isomerases in the therapy and the prophylaxis of neoplastic pathologies.

Protein-disulfide isomerases are a family of enzymes which, in addition to not yet completely elucidated functions, have endoproteinase and chaperonin activities.

The endoproteinase activity can be modulated by calcium ions and is inhibited by Cys-protease inhibitors and is therefore similar to that of other known Cys-proteases, such as calpain, with which no significant sequence homologies exist, apart from the thioredoxin CGHC motif common to many Cys-proteases.

The chaperonin activity has been evidenced in the refolding of the Fab fragment of some monoclonal antibodies and cannot be modulated by calcium ions.

Protein-disulfide isomerases catalyse the formation of disulfide bonds among protein chains and are therefore involved in the “folding” processes of proteins thus playing an important role in the maintenance of fundamental cellular processes.

Examples of protein-disulfide isomerases are the ERp72 endoplasmic reticule proteins (from humans, rat and mouse) described in J. Biol. Chem. 268(29), 22004-22009; 1993; ibidem 269(4), 2501-2507, 1994; ibidem 266, 5353, 1991 and the PS protein from hamster liver described in Biochem. J. 281, 645-650, 1992.

Rupp et al. (J. Biol. Chem. 269(4), 2501-2507, 1994) have defined some of these proteins with the abbreviations CaBP1 and CaBP2, from “Calcium binding protein”.

Other members of the PDI family comprise the proteins known as calsequestrin, BpI, Erp60.

It has now been found that protein-disulfide isomerases (PDI) and related proteins, when injected subcutaneously in Balb/c mice and in rabbits, induce an antibody response of IgM type characterized by a cytotoxicity which can be evidenced in vitro on human tumor cell lines, such as the Jurkat and Kato III lines. The invention therefore relates to the protein-disulfide isomerases as prophylactic and therapeutical agents, in particular as antitumor agents.

The same immunological and cytotoxic properties have been observed in a protein isolated from goat liver with procedures similar to those described for the proteins cited above. Said protein, which has molecular weight of about 72 Kda in SDS-PAGE and a high sequence homology (≧90%) to the ERp72 proteins, is a further object of the invention.

Said protein is obtainable by extraction of mammals liver with PBS followed by purification by chromatography on hydrophobic exchange gel columns.

The process for the extraction and purification of the protein of the invention, in the following referred to as p72, is illustrated in the annexed Figure.

Cytotoxicity is quantifiable in vitro on Jurkat and Kato III cells using conventional methods, based on, for example, the use of commercial kits such as the CDCμK kit (Pharmaproduct). In particular, cytotoxicity was observed in rabbit serum already after a first treatment with p72 (1 mg/animal in saline solution) on Jurkat and Kato III cells.

Cytotoxicity remained steady or increased one month after a second treatment effected two weeks after the first, with values sometimes reaching 85%-89%.

PDIs or p72 are useful as therapeutical or prophylactic agents against tumors of various origin, in particular carcinomas and adenocarcinomas.

PDIs or p72 will be administered at dosages ranging from 1 to 10 mg/patient, using the conventional administration routes for proteins and polypeptides, for example the subcutaneous or intramuscular routes. The treatment can be repeated and a treatment comprising one-two week spaced administrations is preferable.

Furthermore, it has surprisingly been found that high cytotoxicity can also be induced by administering PDIs or p72 at very low dosages, of the order of 1.10⁻⁴-1.10¹⁰⁻¹⁰ g, sublingually, in the form of granules or drops of 1% ethanol water-alcoholic solutions or suspensions, with concentrations of active ingredient ranging from 10⁻⁶ to 10⁻¹⁰ M.

What is claimed:

1. A method of combating tumors in humans, comprising administering to a human in need thereof an anti-tumor effective amount of purified protein-disulfide isomerase in admixture with a pharmaceutically acceptable excipient.

2. The method as claimed in claim 1, wherein said effective amount is 1-10 mg.
